(19) United States
(12) Patent Application Publication
(10) Pub. No.: US 2011/0053979 A1
(43) Pub. Date: Mar. 3, 2011

(54) PYRIDINE DERIVATIVES USED TO TREAT OREXIN RELATED DISORDERS

(76) Inventors: Giuseppe Alvaro, Verona (IT); David Amantini, Verona (IT); Luigi Piero Stasi, Verona (IT)

(21) Appl. No.: 12/936,712

(22) PCT Filed: Apr. 8, 2009
(86) PCT No.: PCT/EP09/54189

§ 371 Date: Oct. 7, 2010

(30) Foreign Application Priority Data
Apr. 10, 2008 (GB) 0806536.9

(51) Int. Cl. A61K 31/4545; C07D 401/12; A61P 25/00; A61P 25/24; A61P 25/30

(52) U.S. Cl. 514/318; 546/194

(57) ABSTRACT

This invention relates to pyridinamine methyl substituted piperidinyl derivatives I and their use as pharmaceuticals.

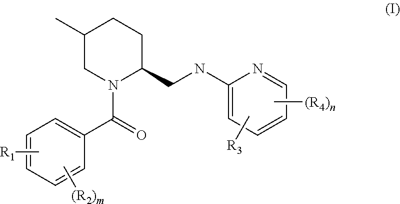

This invention relates to pyridinamine methyl substituted piperidinyl derivatives and their use as pharmaceuticals.

Many medically significant biological processes are mediated by proteins participating in signal transduction pathways that involve G-proteins and/or second messengers.

Polypeptides and polynucleotides encoding the human 7-transmembrane G-protein coupled neuropeptide receptor, orexin-1 (HFGAN72), have been identified and are disclosed in EP875565, EP875566 and WO96/34877. Polypeptides and polynucleotides encoding a second human orexin receptor, orexin-2 (HFGANP), have been identified and are disclosed in EP893498.

Polypeptides and polynucleotides encoding polypeptides which are ligands for the orexin-1 receptor, e.g. orexin-A (Lig72A) are disclosed in EP849361.

The orexin ligand and receptor system has been well characterised since its discovery (see for example Sakurai, T. et al (1998) Cell, 92 pp 573 to 585; Smart et al (1999) British Journal of Pharmacology 128 pp 1 to 3; Willie et al (2001) Ann. Rev. Neurosciences 24 pp 429 to 458; Sakurai (2007) Nature Reviews Neuroscience 8 pp 171 to 181; Ohno and Sakurai (2008) Front. Neuroendocrinology 29 pp 70 to 87). From these studies it has become clear that orexins and orexin receptors play a number of important physiological roles in mammals and open up the possibility of the development of new therapeutic treatments for a variety of diseases and disorders as described hereinbelow.

Experiments have shown that central administration of the ligand orexin-A stimulated food intake in freely-feeding rats during a 4 hour time period. This increase was approximately four-fold over control rats receiving vehicle. These data suggest that orexin-A may be an endogenous regulator of appetite (Sakurai, T. et al (1998) Cell, 92 pp 573 to 585; Peyron et al (1998) J. Neurosciences 18 pp 9996 to 10015; Willie et al (2001) Ann. Rev. Neurosciences 24 pp 429 to 458). Therefore, antagonists of the orexin-A receptor(s) may be useful in the treatment of obesity and diabetes. In support of this it has been shown that orexin receptor antagonist SB334867 potently reduced hedonic eating in rats (White et al (2005) Peptides 26 pp 2231 to 2238) and also attenuated high-fat pellet self-administration in rats (Nair et al (2008) British Journal of Pharmacology, published online 28 Jan. 2008). The search for new therapies to treat obesity and other eating disorders is an important challenge. According to WHO definitions a mean of 35% of subjects in 39 studies were overweight and a further 22% clinically obese in westernised societies. It has been estimated that 5.7% of all healthcare costs in the USA are a consequence of obesity. About 85% of Type 2 diabetics are obese. Diet and exercise are of value in all diabetics. The incidence of diagnosed diabetes in westernised countries is typically 5% and there are estimated to be an equal number undiagnosed. The incidence of both diseases is rising, demonstrating the inadequacy of current treatments which may be either ineffective or have toxicity risks including cardiovascular effects. Treatment of diabetes with sulfonylureas or insulin can cause hypoglycaemia, whilst metformin causes GI side-effects. No drug treatment for Type 2 diabetes has been shown to reduce the long-term complications of the disease. Insulin sensitisers will be useful for many diabetics, however they do not have an anti-obesity effect.

As well as having a role in food intake, the orexin system is also involved in sleep and wakefulness. Rat sleep/EEG studies have shown that central administration of orexin-A, an agonist of the orexin receptors, causes a dose-related increase in arousal, largely at the expense of a reduction in paradoxical sleep and slow wave sleep 2, when administered at the onset of the normal sleep period (Hagan et al (1999) Proc. Natl. Acad. Sci. 96 pp 10911 to 10916). The role of the orexin system in sleep and wakefulness is now well established (Sakurai (2007) Nature Reviews Neuroscience 8 pp 171 to 181; Ohno and Sakurai (2008) Front. Neuroendocrinology 29 pp 70 to 87; Chemelli et al (1999) Cell 98 pp 437 to 451; Lee et al (2005) J. Neuroscience 25 pp 6716 to 6720; Piper et al (2000) European J Neuroscience 12 pp 726-730 and Smart and Jerman (2002) Pharmacology and Therapeutics 94 pp 51 to 61). Antagonists of the orexin receptors may therefore be useful in the treatment of sleep disorders including insomnia. Studies with orexin receptor antagonists, for example SB334867, in rats (see for example Smith et al (2003) Neuroscience Letters 341 pp 256 to 258) and more recently dogs and humans (Brisbare-Roch et al (2007) Nature Medicine 13(2) pp 150 to 155) further support this.

In addition, recent studies have suggested a role for orexin antagonists in the treatment of motivational disorders, such as disorders related to reward seeking behaviours for example drug addiction and substance abuse (Borgland et al (2006) Neuron 49(4) pp 589-601; Boutrel et al (2005) Proc. Natl. Acad. Sci. 102(52) pp 19168 to 19173; Harris et al (2005) Nature 437 pp 556 to 559).

International Patent Applications WO99/09024, WO99/58533, WO00/47577 and WO00/47580 disclose phenyl urea derivatives and WO00/47576 discloses quinolinyl cinnamide derivatives as orexin receptor antagonists. WO05/118548 discloses substituted 1,2,3,4-tetrahydroisoquinoline derivatives as orexin antagonists.

WO01/96302, WO02/44172, WO02/89800, WO03/002559, WO03/002561, WO03/032991, WO03/037847, WO03/041711 and WO08/038,251 all disclose cyclic amine derivatives. In addition WO02/090355 discloses unsubstituted piperidine derivatives and WO04/026866 discloses dialkyl substituted piperidine derivatives as orexin antagonists.

The present invention relates to piperidine derivatives which are monosubstituted with a methyl group at the 5 position on the piperidine ring. The compounds of the invention have been shown to have advantageous properties compared with the compounds in the prior art. These advantages include higher binding affinity to the orexin receptors and an increased bioavailability over prior art compounds when administered in-vivo. Such benefits make the compounds of the invention attractive candidates for use in therapies directed to the treatment of diseases and disorders associated with the orexin system.

The invention provides compounds of formula (I):

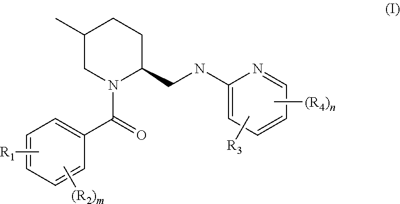

where:
R₁ is C₁₋₄alkyl, halo, C₁₋₄alkoxy, haloC₁₋₄alkyl or haloC₁₋₄alkoxy;
R₂ is C₁₋₄alkyl, halo, C₁₋₄alkoxy, haloC₁₋₄alkyl or haloC₁₋₄alkoxy;
R₃ is C₁₋₄alkyl, halo, C₁₋₄alkoxy, haloC₁₋₄alkyl, haloC₁₋₄alkoxy or cyano;
R₄ is C₁₋₄alkyl, halo, C₁₋₄alkoxy, haloC₁₋₄alkyl or haloC₁₋₄alkoxy; and
m is 0 or 1;
n is 0 or 1;
provided that when m is 1 and one of R₁ and R₂ is trifluoromethyl, the other is not trifluoromethyl;
or a pharmaceutically acceptable derivative salt thereof.

In one embodiment of the present invention the compound has the methyl at the 5 position on the piperidyl ring in the 5S isomeric form. In another embodiment the compound has the methyl at the 5 position on the piperidyl ring in the 51? isomeric form. In a further embodiment the compound has a racemic mixture of both 5S and 5R isomeric forms.

In one embodiment n is 0.

In another embodiment n is 1.

In one embodiment m is 0.

In another embodiment m is 1.

In one embodiment R₁ is C₁₋₄alkyl. In another embodiment R₁ is methyl.

In one embodiment R₁ is C₁₋₄alkoxy. In another embodiment R₁ is methyloxy, ethyloxy or propyloxy.

In one embodiment R₁ is halo. In another embodiment R₁ is fluoro or chloro.

In one embodiment R₁ is haloC₁₋₄alkoxy. In another embodiment R₁ is trifluoromethyloxy.

In one embodiment m is 1, R₁ is C₁₋₄alkoxy and R₂ is C₁₋₄alkoxy. In another embodiment m is 1, R₁ is ethyloxy and R₂ is ethyloxy.

In one embodiment m is 1, R₁ is C₁₋₄alkoxy and R₂ is halo. In another embodiment m is 1, R₁ is methyloxy and R₂ is fluoro. In a further embodiment m is 1, R₁ is methyloxy and R₂ is chloro. In a still further embodiment m is 1, R₁ is ethyloxy and R₂ is chloro. In a yet further embodiment m is 1, R₁ is 2-methylpropyloxy and R₂ is chloro. In a still further embodiment m is 1, R₁ is ethyloxy and R₂ is fluoro. In a yet further embodiment m is 1, R₁ is propyloxy and R₂ is fluoro.

In one embodiment m is 1, R₁ is methyloxy which is attached at the 2 position on the phenyl ring and R₂ is fluoro which is attached at the 3 position on the phenyl ring.

In one embodiment R₃ is halo. In another embodiment R₃ is bromo, chloro or fluoro. In a still further embodiment R₃ is bromo.

In one embodiment R₃ is C₁₋₄alkyl. In another embodiment R₃ is methyl.

In one embodiment R₃ is cyano.

In one embodiment R₃ is haloC₁₋₄alkyl. In another embodiment R₃ is trifluoromethyl.

In a further embodiment R₃ is attached at the 5 position of the pyridyl ring.

In one embodiment n is 1 and R₄ is C₁₋₄alkyl. In another embodiment n is 1 and R₄ is methyl.

In one embodiment n is 1 and R₄ is halo. In another embodiment n is 1 and R₄ is fluoro.

In one embodiment n is 1, R₃ is halo and R₄ is C₁₋₄alkyl. In another embodiment n is 1, R₃ is fluoro and R₄ is methyl.

In one embodiment n is 1, R₃ is halo and R₄ is halo. In another embodiment n is 1, R₃ is fluoro and R₄ is fluoro.

In one embodiment m is 1, n is 0, R₁ is C₁₋₄alkoxy, R₂ is halo and R₃ is halo. In another embodiment m is 1, n is 0, R₁ is methyloxy, R₂ is fluoro and R₃ is bromo or chloro. In one embodiment m is 1, n is 0, R₁ is C₁₋₄alkoxy, R₂ is halo and R₃ is haloC₁₋₄alkyl. In another embodiment m is 1, n is 0, R₁ is methyloxy, R₂ is fluoro and R₃ is trifluoromethyl.

In a another embodiment m is 1, n is 0, R₁ is methyloxy which is attached at the 2 position on the phenyl ring, R₂ is fluoro which is attached at the 3 position on the phenyl ring and R₃ is bromo which is attached at the 5 position of the pyridyl ring.

In a further embodiment m is 1, n is 0, R₁ is methyloxy which is attached at the 2 position on the phenyl ring, R₂ is fluoro which is attached at the 3 position on the phenyl ring and R₃ is trifluoromethyl which is attached at the 5 position of the pyridyl ring.

In a still further embodiment m is 1, n is 0, R₁ is methyloxy which is attached at the 2 position on the phenyl ring, R₂ is fluoro which is attached at the 3 position on the phenyl ring and R₃ is chloro which is attached at the 5 position of the pyridyl ring.

Examples of compounds of the invention include:
N-[((2S,5S)-1-{[2,6-bis(ethyloxy)phenyl]carbonyl}-5-methyl-2-piperidinyl)methyl]-5-bromo-2-pyridinamine;
5-bromo-N-[((2S,5S)-5-methyl-1-{[2-(methyloxy)phenyl]carbonyl}-2-piperidinyl)methyl]-2-pyridinamine;
5-bromo-N-[((2S,5S)-1-{[2-(ethyloxy)phenyl]carbonyl}-5-methyl-2-piperidinyl)methyl]-2-pyridinamine;
5-bromo-N-[((2S,5S)-1-{[3-fluoro-2-(methyloxy)phenyl]carbonyl}-5-methyl-2-piperidinyl)methyl]-2-pyridinamine;
5-bromo-N-[((2S,5S)-1-{[4-chloro-2-(methyloxy)phenyl]carbonyl}-5-methyl-2-piperidinyl)methyl]-2-pyridinamine;
5-bromo-N-{[(2S,5S)-5-methyl-1-({2-[(trifluoromethyl)oxy]phenyl}carbonyl)-2-piperidinyl]methyl}-2-pyridinamine;
5-bromo-N-({(2 S,5S)-5-methyl-1-[(2-methylphenyl)carbonyl]-2-piperidinyl}methyl)-2-pyridinamine;
N-[((2S,5S)-1-{[3-fluoro-2-(methyloxy)phenyl]carbonyl}-5-methyl-2-piperidinyl)methyl]-6-methyl-2-pyridinamine hydrochloride;
6-methyl-N-{[(2S,5S)-5-methyl-1-({2-[(trifluoromethyl)oxy]phenyl}carbonyl)-2-piperidinyl]methyl}-2-pyridinamine;
5-bromo-N-[((2S,5S)-1-{[5-fluoro-2-(methyloxy)phenyl]carbonyl}-5-methyl-2-piperidinyl)methyl]-2-pyridinamine;
5-bromo-N-[((2S,5S)-1-{[4-fluoro-2-(methyloxy)phenyl]carbonyl}-5-methyl-2-piperidinyl)methyl]-2-pyridinamine;
5-bromo-N-[((2S,5S)-1-{[2-fluoro-6-(methyloxy)phenyl]carbonyl}-5-methyl-2-piperidinyl)methyl]-2-pyridinamine;
N-{[(2S,5S)-1-({5-chloro-2-[(2-methylpropyl)oxy]phenyl}carbonyl)-5-methyl-2-piperidinyl]methyl}-6-methyl-2-pyridinamine hydrochloride;
N-[((2S,5S)-1-{[5-chloro-2-(ethyloxy)phenyl]carbonyl}-5-methyl-2-piperidinyl)methyl]-6-methyl-2-pyridinamine hydrochloride;
6-{[((2S,5S)-1-{[3-fluoro-2-(methyloxy)phenyl]carbonyl}-5-methyl-2-piperidinyl)methyl]amino}-3-pyridinecarbonitrile;
N-[((2S,5S)-1-{[3-fluoro-2-(methyloxy)phenyl]carbonyl}-5-methyl-2-piperidinyl)methyl]-5-(trifluoromethyl)-2-pyridinamine;
N-[((2S,5S)-1-{[3-fluoro-2-(methyloxy)phenyl]carbonyl}-5-methyl-2-piperidinyl)methyl]-6-(trifluoromethyl)-2-pyridinamine;
N-[((2S,5S)-1-{[3-fluoro-2-(methyloxy)phenyl]carbonyl}-5-methyl-2-piperidinyl)methyl]-3-(trifluoromethyl)-2-pyridinamine;
3,5-difluoro-N-[((2S,5S)-1-{[3-fluoro-2-(methyloxy)phenyl]carbonyl}-5-methyl-2-piperidinyl)methyl]-2-pyridinamine;
N-[((2S,5S)-1-{[3-fluoro-2-(methyloxy)phenyl]carbonyl}-5-methyl-2-piperidinyl)methyl]-4-(trifluoromethyl)-2-pyridinamine;
N-[((2S,5S)-1-{[3-fluoro-2-(methyloxy)phenyl]carbonyl}-5-methyl-2-piperidinyl)methyl]-5-methyl-2-pyridinamine;
5-chloro-N-[((2S,5S)-1-{[3-fluoro-2-(methyloxy)phenyl]carbonyl}-5-methyl-2-piperidinyl)methyl]-2-pyridinamine;
5-fluoro-N-[((2S,5S)-1-{[3-fluoro-2-(methyloxy)phenyl]carbonyl}-5-methyl-2-piperidinyl)methyl]-4-methyl-2-pyridinamine;
5-ethyl-N-[((2S,5S)-1-{[3-fluoro-2-(methyloxy)phenyl]carbonyl}-5-methyl-2-piperidinyl)methyl]-2-pyridinamine;
5-chloro-N-[((2S,5S)-1-{[2-(ethyloxy)-3-fluorophenyl]carbonyl}-5-methyl-2-piperidinyl)methyl]-2-pyridinamine; and
N-[((2S,5S)-1-{[3-fluoro-2-(propyloxy)phenyl]carbonyl}-5-methyl-2-piperidinyl)methyl]-5-methyl-2-pyridinamine.

The compounds of the invention are (25) diastereoisomers. The stereochemistry of the methyl at the 5 position on the piperidine may be either (5R) or (55), although the racemic form of the compounds (ie. having both (5R) or (55) configurations) are included within the scope of the invention. If required the different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

It will be understood that the invention includes pharmaceutically acceptable derivatives of compounds of formula (I) and that these are included within the scope of the invention.

Particular compounds according to the invention include those mentioned in the examples and their pharmaceutically acceptable derivatives.

As used herein “pharmaceutically acceptable derivative” includes any pharmaceutically acceptable salt, ester or salt of such ester of a compound of formula (I) which, upon administration to the recipient is capable of providing (directly or indirectly) a compound of formula (I) or an active metabolite or residue thereof.

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge, Bighley and Monkhouse J. Pharm. Sci (1977) 66, pp1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention. Also included within the scope of the invention are solvates and hydrates of compounds of formula (I).

Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form and, if crystalline, may optionally be solvated, eg. as the hydrate. This invention includes within its scope stoichiometric solvates (eg. hydrates) as well as compounds containing variable amounts of solvent (eg. water).

The subject invention also includes isotopically-labeled compounds which are identical to those recited in formula (I) and following, but for the fact that one or more atoms are replaced by an atom having an atomic mass or mass number different from the atomic mass or mass number most commonly found in nature. Examples if isotopes that can be incorporated into compounds of the invention include isotopes of hydrogen, carbon, nitrogen, oxygen, fluorine, iodine and chlorine such as ³H, ¹¹C, ¹⁴C, 18F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, ie. ³H, and carbon-14, ie. ¹⁴C, isotopes are particularly preferred for their ease of preparation and detectability. ¹¹C and ¹⁸F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further aspect of the present invention there is provided a process for the preparation of compounds of formula (I) and derivatives thereof. The following schemes detail some synthetic routes to compounds of the invention. In the following schemes reactive groups can be protected with protecting groups and deprotected according to well established techniques.

### Schemes

According to a further feature of the invention there is provided a process for the preparation of compounds of formula (I) and derivatives thereof. The following is an example of a synthetic scheme that may be used to synthesise the compounds of the invention.

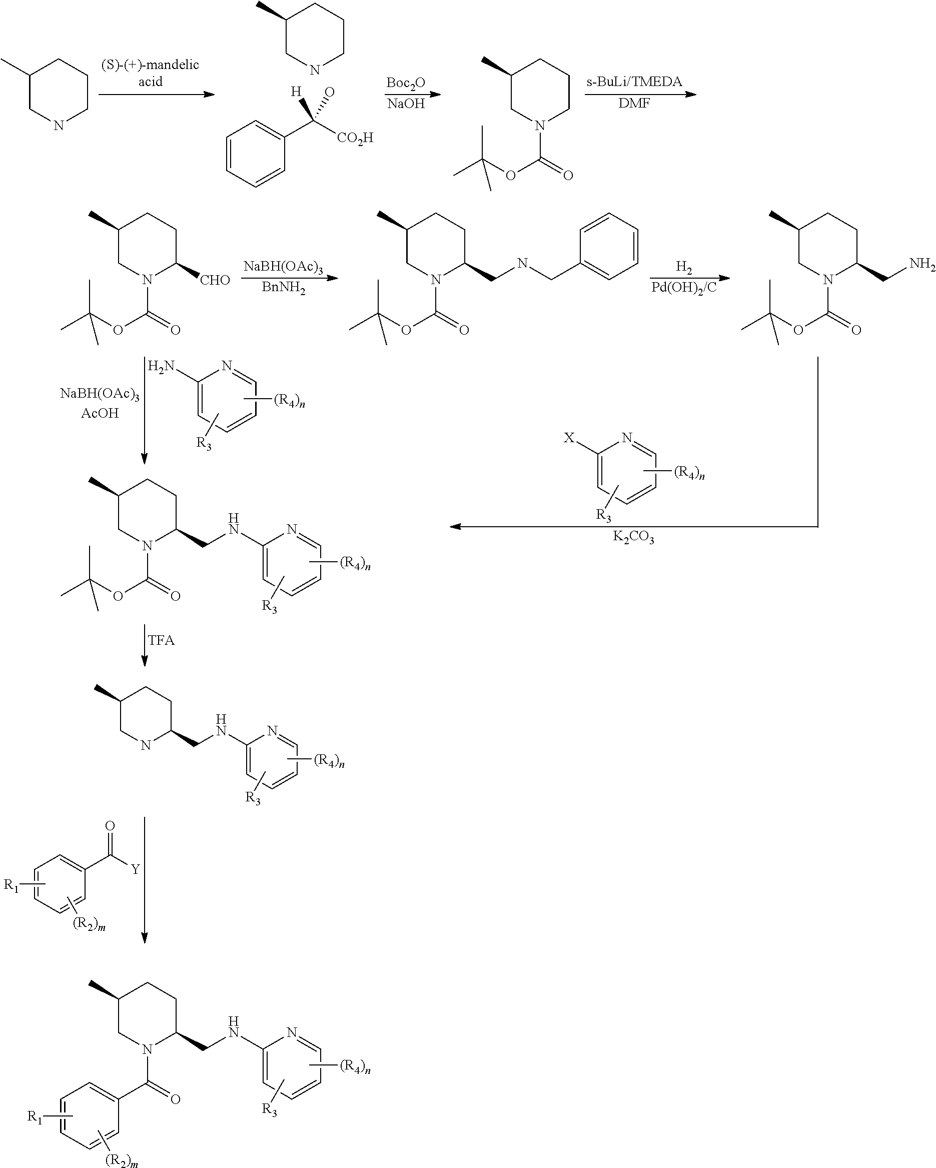

It will be understood by those skilled in the art that certain compounds of the invention can be converted into other compounds of the invention according to standard chemical methods.

The starting materials for use in the scheme are commercially available, known in the literature or can be prepared by known methods.

The compounds of formula (I) may be prepared singly or as compound libraries comprising at least 2, e.g. 5 to 1000, preferably 10 to 100 compounds of formula (I). Compound libraries may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I), or pharmaceutically acceptable derivatives thereof.

Pharmaceutically acceptable salts may be prepared conventionally by reaction with the appropriate acid or acid derivative.

The present invention provides compounds of formula (I) and their pharmaceutically acceptable derivatives for use in human or veterinary medicine.

The compounds of formula (I) and their pharmaceutically acceptable derivatives may be of use for the treatment of diseases or disorders where an antagonist of a human orexin receptor is required such as sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In addition the compounds of formula (I) and their pharmaceutically acceptable derivatives may be of use for the treatment of diseases or disorders where an antagonist of a human orexin receptor is required such as depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar 1Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

Further, the compounds of formula (I) and their pharmaceutically acceptable derivatives may be of use for the treatment of diseases or disorders where an antagonist of a human orexin receptor is required such as anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00).

In addition the compounds of formula (I) and their pharmaceutically acceptable derivatives may be of use for the treatment of diseases or disorders where an antagonist of a human orexin receptor is required such as substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

In addition the compounds of formula (I) and their pharmaceutically acceptable derivatives may be of use for the treatment of diseases or disorders where an antagonist of a human orexin receptor is required such as feeding disorders such as Eating disorders include Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50).

The numbers in brackets after the listed diseases refer to the classification code in DSM-IV: Diagnostic and Statistical Manual of Mental Disorders, 4th Edition, published by the American Psychiatric Association. The various subtypes of the disorders mentioned herein are contemplated as part of the present invention.

The invention also provides a method of treating or preventing diseases or disorders where an antagonist of a human orexin receptor is required, for example those diseases and disorders mentioned hereinabove, which comprises administering to a subject in need thereof an effective amount of a compound of formula (I) or a pharmaceutically acceptable derivative thereof.

The invention also provides a compound of formula (I), or a pharmaceutically acceptable derivative thereof, for use in the treatment or prophylaxis of diseases or disorders where an antagonist of a human orexin receptor is required, for example those diseases and disorders mentioned hereinabove.

The invention also provides the use of a compound of formula (I), or a pharmaceutically acceptable derivative thereof, in the manufacture of a medicament for the treatment or prophylaxis of diseases or disorders where an antagonist of a human orexin receptor is required, for example those diseases and disorders mentioned hereinabove.

For use in therapy the compounds of the invention are usually administered as a pharmaceutical composition. The invention also provides a pharmaceutical composition comprising a compound of formula (I), or a pharmaceutically acceptable derivative thereof, and a pharmaceutically acceptable carrier.

The compounds of formula (I) and their pharmaceutically acceptable derivatives may be administered by any convenient method, e.g. by oral, parenteral, buccal, sublingual, nasal, rectal or transdermal administration, and the pharmaceutical compositions adapted accordingly.

The compounds of formula (I) and their pharmaceutically acceptable derivatives which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

Preferably the composition is in unit dose form such as a tablet, capsule or ampoule.

The dose of the compound of formula (I), or a pharmaceutically acceptable derivative thereof, used in the treatment or prophylaxis of the above-mentioned disorders or diseases will vary in the usual way with the particular disorder or disease being treated, the weight of the subject and other similar factors. However, as a general rule, suitable unit doses may be 0.05 to 1000 mg, more suitably 0.05 to 500 mg. Unit doses may be administered more than once a day for example two or three times a day, so that the total daily dosage is in the range of about 0.01 to 100 mg/kg. Such therapy may extend for a number of weeks or months. In the case of pharmaceutically acceptable derivatives the above figures are calculated as the parent compound of formula (I).

No toxicological effects are indicated/expected when a compound of formula (I) is administered in the above mentioned dosage range.

Orexin-A (Sakurai, T. et al (1998) Cell, 92 pp 573-585) can be employed in screening or assay procedures for compounds which inhibit the ligand's activation of the orexin-1 receptor.

In general, such screening or assay procedures involve providing appropriate cells which express the orexin-1 receptor on their surface. Such cells include cells from mammals, yeast, Drosophila or E. coli. In particular, a polynucleotide encoding the orexin-1 receptor is used to transfect cells to express the receptor. The expressed receptor is then contacted with a test compound and an orexin-1 receptor ligand to observe inhibition of a functional response. One such screening or assay procedure involves the use of melanophores which are transfected to express the orexin-1 receptor, as described in WO 92/01810.

Another screening or assay procedure involves introducing RNA encoding the orexin-1 receptor into Xenopus oocytes to transiently express the receptor. The receptor oocytes are then contacted with a receptor ligand and a test compound, followed by detection of inhibition of a signal in the case of screening for compounds which are thought to inhibit activation of the receptor by the ligand.

Another method involves screening for compounds which inhibit activation of the receptor by determining inhibition of binding of a labelled orexin-1 receptor ligand to cells which have the receptor on their surface. This method involves transfecting a eukaryotic cell with DNA encoding the orexin-1 receptor such that the cell expresses the receptor on its surface and contacting the cell or cell membrane preparation with a compound in the presence of a labelled form of an orexin-1 receptor ligand. The ligand may contain a radioactive label. The amount of labelled ligand bound to the receptors is measured, e.g. by measuring radioactivity.

Yet another screening technique involves the use of FLIPR equipment for high throughput screening of test compounds that inhibit mobilisation of intracellular calcium ions, or other ions, by affecting the interaction of an orexin-1 receptor ligand with the orexin-1 receptor.

Throughout the specification and claims which follow, unless the context requires otherwise, the word ‘comprise’, and variations such as ‘comprises’ and ‘comprising’ will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The following Examples illustrate the preparation of pharmacologically active compounds of the invention. The Descriptions 1 to 11 illustrate the preparation of intermediates to compounds of the invention.

In the procedures that follow, after each starting material, reference to a description is typically provided. This is provided merely for assistance to the skilled chemist. The starting material may not necessarily have been prepared from the Description referred to.

The yields were calculated assuming that products were 100% pure if not stated otherwise.

The compounds described in the Examples have all been prepared from stereochemically pure (minimum enantiomeric purity 94%) 1,1-dimethylethyl (3S)-3-methyl-1-piperidinecarboxylate (D2). The stereochemistry of the compounds of the Descriptions and Examples have been assigned on the assumption that the pure configuration of this centre is retained.

Compounds are named using ACD/Name PRO6.02 chemical naming software (Advanced Chemistry Development Inc., Toronto, Ontario, M5H2L3, Canada).

Proton Magnetic Resonance (NMR) spectra were recorded either on Varian instruments at 400, 500 or 600 MHz, or on Bruker instruments at 400 MHz. Mono-(1H and 1H with homonuclear decoupling) and two-dimensional techniques (1H-1H COSY, 1H-1H ROESY, 1H-13C HSQC) were used for stereochemistry investigation. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is usually reported.

HPLC analysis indicated by Rt(HPLC): x min, was performed on an Agilent 1100 series instrument using a Luna 3u C18(2) 100A (50×2.0 mm) column (mobile phase: 100% [water+0.05% TFA] to 95% [acetonitrile+0.05% TFA] in 8 min, flux=1 ml/min, detection wavelength 220 nm.

Mass spectra (MS) were taken on a 4 II triple quadrupole Mass Spectrometer (Micromass UK) or on a Agilent MSD 1100 Mass Spectrometer, operating in ES (+) and ES (−) ionization mode or on an Agilent LC/MSD 1100 Mass Spectrometer, operating in ES (+) and ES (−) ionization mode coupled with HPLC instrument Agilent 1100 Series [LC/MS-ES (+): analysis performed on a Supelcosil ABZ+Plus (33×4.6 mm, 3 μm) (mobile phase: 100% [water+0.1% HCO₂H] for 1 min, then from 100% [water+0.1% HCO₂H] to 5% [water+0.1% HCO₂H] and 95% [CH₃CN] in 5 min, finally under these conditions for 2 min; T=40° C.; flux=1 mL/min; LC/MS-ES (−): analysis performed on a Supelcosil ABZ+Plus (33×4.6 mm, 3 μm) (mobile phase: 100% [water+0.05% NH₃] for 1 min, then from 100% [water+0.05% NH₃ to 5% [water+0.05% NH₃] and 95% [CH₃CN] in 5 min, finally under these conditions for 2 min; T=40° C.; flux=1 mL/min]. In the mass spectra only one peak in the molecular ion cluster is reported.

Total ion current (TIC) and DAD UV chromatographic traces together with MS and UV spectra associated with the peaks were taken also on a UPLC/MS Acquity™ system equipped with 2996 PDA detector and coupled to a Waters Micromass ZQTM mass spectrometer operating in positive or negative electrospray ionisation mode. [LC/MS-ES (+/−): analyses performed using an Acquity™ UPLC BEH C18 column (50×21 mm, 1.7 μm particle size), column temperature 40° C. (mobile phase: A-water+0.1% HCOOH/B-CH₃CN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)]. The usage of this methodology is indicated by “UPLC” in the analytic characterization of the described compounds.

Preparative LC-MS purifications were performed on a MDAP (Mass Detector Auto Purification) Waters instrument. The usage of this methodology is indicated by “Fraction Lynx” in the analytic characterization of the described compounds. [LC3_{—}100 mg method. Column: Waters XTerra Prep MS C18 OBD, 30×150 mm, 10 μm particle size. Mobile phase: A: H₂O+0.1% formic acid; B: CH₃CN+0.1% formic acid. Flow rate 40 ml/min. Gradient: 30% to 55% (B) in 10 min, 55% to 99% (B) in 4 min, 95% to 100% (B) in 1 min. UV range: 210-400 nm. Ionization: ES+/ES−. Mass range: 150-900 amu].

For reactions involving microwave irradiation, a Personal Chemistry Emrys™ Optimizer was used.

In a number of preparations, purification was performed using Biotage manual flash chromatography (Flash+), Biotage automatic flash chromatography (Horizon, SP1 and SP4), Companion CombiFlash (ISCO) automatic flash chromatography, Flash Master Personal or Vac Master systems.

Flash chromatography was carried out on silica gel 230-400 mesh (supplied by Merck AG Darmstadt, Germany), Varian Mega Be—Si pre-packed cartridges, pre-packed Biotage silica cartridges (e.g. Biotage SNAP cartridge), KP—NH prepacked flash cartridges or ISCORediSep Silica cartridges.

SPE-SCX cartridges are ion exchange solid phase extraction columns supplied by Varian. The eluent used with SPE-SCX cartridges is methanol followed by 2N ammonia solution in methanol.

SPE-Si cartridges are silica solid phase extraction columns supplied by Varian.

The following table lists the used abbreviations:

AcCl Acetyl chloride

### ACN Acetonitrile

AcOH Acetic acid
atm atmosphere
bs broad signal
Boc t-Butoxycarbonyl

### BnNH₂ Benzylamine

n-BuLi n-Butyl Lithium
s-BuLi s-Butyl Lithium

### Cy Cyclohexanes

### DCE 1,2-Dichloroethane

### DCM Dichloromethane

### DIPEA N,N-diisopropyl-N-ethylamine

### DMF N,N-Dimethylformamide

### DMSO Dimethylsulfoxide

### Et₂O Diethylether

### EtOAc Ethylacetate

HATU (2-(7-Aza-1H-benzotriazole-1-yl)-1,1,3,3-tetramethyluronium hexafluorophosphate)

### MeOH Methanol

rt room temperature
TBTU O-(benzotriazol-1-yl)-N,N,N′N′-tetramethyluronium tetrafluoroborate
TFA Trifluoroacetic acid

### THF Tetrahydrofuran

### TMEDA N,N,N′,N′-Tetramethylethylendiamine

### DESCRIPTIONS

### Description 1

### (2S)-hydroxy(phenyl)ethanoic acid-(3S)-3-methylpiperidine (1:1) (D1)

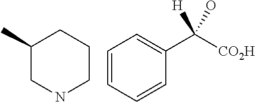

In a 10 L reactor, under nitrogen atmosphere, a solution of racemic 3-methylpiperidine (270 g, 2.72 mol) and (S)-(+)-mandelic acid (394 g, 2.59 mol) in MeOH (1 L) was cooled to 0° C. Without stirring, Et₂O (6.21 L) was added in several portions: (10×540 ml) every 20 minutes and 810 ml after 30 minutes from the last addition. After each addition of Et₂O, a short and slow stirring was applied in order to obtain an homogeneous phase. The final slurry was left standing overnight at 0° C. The precipitated solid was recovered by filtration, washed with cold Et₂O (2×540 ml) and dried under vacuum to afford the title compound D1 (150 g, 0.60 mol, 23% yield) [optical purity (94%) was determined by preparation of the Mosher amide derivative. The diastereomeric excess of the Mosher amide, determined via NMR spectroscopy, is representative of the enantiomeric excess of the precursor].

¹H-NMR (400 MHz, CDCl₃) δ (ppm): 7.43-7.50 (m, 2H), 7.20-7.34 (m, 3H), 4.89 (s, 1H), 2.89-3.05 (m, 2H), 2.17 (dt, 1H), 2.06 (t, 1H), 1.39-1.73 (m, 4H), 0.83-0.98 (m, 1H), 0.80 (d, 3H).

### Description 2

### 1,1-dimethylethyl (3S)-3-methyl-1-piperidinecarboxylate (D2)

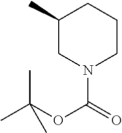

To a mixture of (2S)-hydroxy(phenyl)ethanoic acid-(3S)-3-methylpiperidine (1:1) (D1) (150 g, 0.60 mol) in a 2.5 M NaOH aqueous solution (600 ml, 1.50 mol) cooled at
0° C., a solution of Boc₂O (130 g, 0.60 mol) in THF (1.2 L) was added dropwise over 1 hour (internal temperature kept below 9° C.) under vigorous stirring. Once the addition was completed, the mixture was allowed to reach rt and left under stirring overnight. Volatiles were evaporated and the aqueous phase extracted with Et₂O (3×500 ml). The collected organic phases were dried (Na₂SO₄), filtered and concentrated to dryness. The resulting crude material was eluted (Cy/EtOAc 90/10) through a silica gel pad to give the title compound D2 (103 g, 0.52 mol, 87% yield). UPLC: rt=0.85 min, peak observed: 200 (M+1). C₁₁H₂₁NO₂ requires 199.

¹H NMR (400 MHz, CDCl₃) δ (ppm): 3.95 (bd, 2H), 2.70 (dt, 1H), 2.21-2.55 (m, 1 H), 1.73-1.86 (m, 1H), 1.51-1.68 (m, 3H), 1.47 (s, 9H), 0.96-1.12 (m, 1H), 0.88 (d, 3H).

### Description 3

### 1,1-dimethylethyl (2S,5S)-2-formyl-5-methyl-1-piperidinecarboxylate (D3)

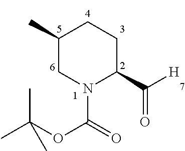

To a solution of 1,1-dimethylethyl (3S)-3-methyl-1-piperidinecarboxylate (D2) (25 g, 0.13 mol) in anhydrous Et₂O (250 ml) cooled at −78° C. under nitrogen atmosphere, TMEDA (22.6 ml, 0.15 mol) was added followed by dropwise addition of s-BuLi (108 ml of a 1.4 M solution in Cy, 0.15 mol) over 40 min (exothermic addition: internal temperature kept below −70° C.). The pale yellow reaction mixture was left under stirring at −78° C. for 30 min then it was gradually warmed to −50° C. and stirred at this temperature for 30 min. The reaction was cooled again to −78° C., then TMEDA (further 0.3 eq) was added followed by dropwise addition of s-BuLi (further 0.3 eq). The mixture was stirred for 30 min at −78° C., gradually warmed up to −50° C., stirred at this temperature for 30 min, then cooled to −78° C. Dry DMF (29.1 ml, 0.38 mol) was added dropwise (internal temperature kept below −70° C.). The resulting mixture was stirred for 30 minutes at −78° C. and then allowed to warm up to 0° C. The reaction mixture was quenched with a saturated NH₄Cl aqueous solution (200 ml) and water (100 ml). The layers were separated and the aqueous one back extracted with Et₂O (3×200 ml). The organic phases were collected, dried (Na₂SO₄), filtered and concentrated under vacuum to give a crude yellow oil. The material was purified by flash chromatography on silica gel (Biotage 75L, Cy/EtOAc 90/10). Collected fractions gave the title compound D3 (15 g, 0.066 mol, 53% yield).

¹H NMR [the relative stereochemistry of the compound was measured via NMR spectroscopy. The 1H spectrum shows that the compound gives rise to a mixture of two slowly exchanging conformers due to hindered rotation of the C═O group. 1H,1H scalar couplings [³J(H3,H2)-5 Hz and ³J(H6ax,H5ax)-12 Hz] and 1H,1H dipole dipole correlation between H7 and H4ax determine that the six member ring bears a chair conformation with H2 in equatorial position and H5 in axial position. The relative stereochemistry is therefore SYN. The ANTI stereoisomer is present at ca. 25%. The ratio between the two diastereoisomers was determined on the ratio between integrals of proton signals H7 of each diastereoisomer. The absolute configuration is 2S,5S on the assumption that the absolute configuration of D2 is retained. The assignment refers to the SYN isomer] (400 MHz, DMSO-d₆) δ (ppm): 9.53 (d, 1H), 4.53-4.72 (m, 1H), 3.73-3.91 (m, 1H), 2.39 (t, 1H), 2.16-2.27 (m, 1H), 1.52-1.72 (m, 3H), 1.40 (s, 9H), 0.80 (d, 3H), 0.68-0.77 (m, 1H).

### Description 4

### 1,1-dimethylethyl (2S,5S)-5-methyl-2-{[(phenylmethyl)amino]methyl}-1-piperidinecarboxylate (D4)

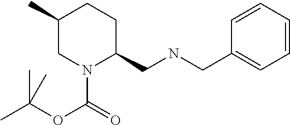

A solution of 1,1-dimethylethyl (2S,5S)-2-formyl-5-methyl-1-piperidinecarboxylate (D3) (0.45 g, 1.98 mmol) and benzylamine (0.24 ml, 2.18 mmol) in DCM (5 ml) was left under stirring at rt under nitrogen for 2 h. Sodium triacetoxyborohydride (0.84 g, 3.96 mmol) was added and the resulting solution left under stirring at rt overnight. The mixture was diluted with water and DCM. The two layers were separated and the aqueous one extracted several times with DCM. The combined organic layers were filtered through a phase separator tube and the solvent removed under vacuum. The crude was purified by flash chromatography on silica gel (Biotage SP4 40M, from Cy/EtOAc from 80/20 to 20/80). Collected fractions gave the title compound D4 (0.37 g, 1.16 mmol, 59% yield). HPLC (walk-up): rt=3.86 min.

¹H NMR [the SYN relative stereochemistry is derived from 1H,1H scalar coupling network. A mixture of conformers due to hindered rotation of the C═O group slowly exchange in solution. Only one rotamer is assigned] (400 MHz, CDCl₃) δ (ppm): 7.31-7.36 (m, 4H), 7.23-7.27 (m, 1H), 4.23-4.49 (m, 1H), 3.70-4.09 (m, 1H), 3.87 (d, 1H), 3.79 (d, 1H), 2.89 (dd, 1H), 2.62 (dd, 1H), 2.21-2.39 (m, 1H), 1.53-1.75 (m, 4H), 1.47 (s, 9H), 1.06-1.18 (m, 1H), 0.87 (d, 3H).

### Description 5

### 1,1-dimethylethyl (2S,55)-2-(aminomethyl)-5-methyl-1-piperidinecarboxylate (D5)

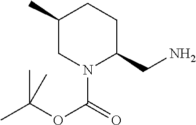

A mixture of 1,1-dimethylethyl (2S,5S)-5-methyl-2-{[(phenylmethyl)amino]methyl}-1-piperidinecarboxylate (D4) (0.37 g, 1.16 mmol) and Pd(OH)₂ on carbon (0.011 g) in MeOH (5 ml) was stirred under hydrogen atmosphere (1 atm) for 27 h. Further Pd(OH)₂ on carbon (0.011 g) was added and the resulting mixture left under stirring under hydrogen atmosphere (1 atm) for 7 h. The mixture was filtered through a celite pad and the solvent evaporated under vacuum to afford the title compound D5 (0.24 g, 1.05 mmol, 91% yield) as a yellow oil. UPLC: rt=0.50 min, peak observed: 229 (M+1). C₁₂H₂₄N₂O₂ requires 228. ¹H NMR [the SYN relative stereochemistry is derived from 1H,1H scalar coupling network. A mixture of conformers due to hindered rotation of the C═O group slowly exchange in solution. Only one rotamer is assigned] (400 MHz, CDCl₃) δ (ppm): 3.75-4.31 (m, 2H), 2.84-2.99 (m, 1H), 2.61-2.71 (m, 1H), 2.24-2.42 (m, 1H), 1.50-1.72 (m, 4H), 1.48 (s, 9H), 1.07-1.22 (m, 1H), 0.89 (d, 3H).

### Description 6

### 1,1-dimethylethyl (2S,5S)-2-{[(5-bromo-2-pyridinyl)amino]methyl}-5-methyl-1-piperidinecarboxylate (D6)

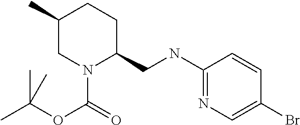

In a 1 L round-bottomed flask 1,1-dimethylethyl (2S,5S)-2-formyl-5-methyl-1-piperidinecarboxylate (13.0 g, 57.2 mmol) (D3) and 5-bromo-2-pyridinamine (11.9 g, 68.6 mmol) were dissolved in DCE (390 ml) at rt under nitrogen atmosphere. AcOH (16.4 ml, 286 mmol) was added, followed by sodium triacetoxyborohydride (14.6 g, 68.6 mmol) after 1 h. The reaction mixture was stirred for 64 h at rt. A saturated NaHCO₃ aqueous solution (400 ml) and DCM (400 ml) were added and the phases separated. The aqueous phase was back extracted with DCM (2×400 ml) and the combined organic layers were dried (Na₂SO₄). The solution was then concentrated to dryness under vacuum to get a yellow oil. This material was combined with 2.3 g of a crude coming from an identical reaction carried out on 1 g (4.4 mmol) of D3 and purified by flash chromatography on silica gel (Biotage 75L, Cy/EtOAc 90/10). Collected fractions gave the title compound D6 (10.6 g, 27.6 mmol, 45% yield) as pale yellow foam. MS: (ES/+) m/z: 384 (M+1, 100%) and 386 (M+1, 100%). C₁₋₇H₂₆BrN₃O₂ requires 383.

### Description 7

### 5-bromo-N-{[(2S,5S)-5-methyl-2-piperidinyl]methyl}-2-pyridinamine (D7)

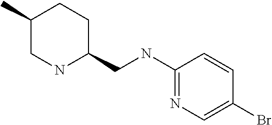

To a solution of 1,1-dimethylethyl (2S,5S)-2-{[(5-bromo-2-pyridinyl)amino]methyl}-5-methyl-1-piperidinecarboxylate (D6) (10.6 g, 27.6 mmol) in DCM (85 ml), TFA (21.3 ml, 276 mmol) was added dropwise at 0° C. under nitrogen atmosphere. After the addition, the reaction mixture was allowed to warm up to room temperature and left under stirring for 2.5 h at this temperature. Solvent was evaporated under vacuum at rt and the crude slurry was diluted with DCM (200 ml) and a saturated K₂CO₃ aqueous solution (80 ml). Phases were separated and the aqueous layer back-extracted with DCM (2×150 ml). The combined organic layers were washed with brine (1×50 ml). The organic phase was dried (Na₂SO₄), filtered and concentrated to get the title compound D7 (7.82 g, 27.5 mmol, quantitative yield) as a yellow oil.

¹H-NMR [the SYN relative stereochemistry is derived from 1H,1H scalar coupling network] (400 MHz, CDCl₃) δ (ppm): 8.06-8.09 (m, 1H), 7.41 (dd, 1H), 6.32 (d, 1 H), 4.98-5.20 (bs, 1H), 3.10-3.39 (m, 2H), 2.85-2.97 (m, 1H), 2.80 (dd, 1H), 2.65 (dd, 1H), 1.40-1.78 (m, 5H), 0.97 (d, 3H).

### Description 8

### 1,1-dimethylethyl (2S,5S)-5-methyl-2-({[5-(trifluoromethyl)-2-pyridinyl]amino}methyl)-1-piperidinecarboxylate (D8)

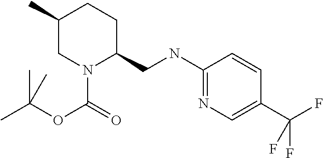

A mixture of 1,1-dimethylethyl (2S,55)-2-(aminomethyl)-5-methyl-1-piperidinecarboxylate (D5) (0.13 g, 0.57 mmol), 2-chloro-5-(trifluoromethyl)pyridine (0.10 g, 0.57 mmol) and potassium carbonate (0.16 g, 1.14 mmol) in DMF (2 ml) was stirred at 80° C. for 5 h. DMF was removed under reduced pressure. The residue was taken-up in DCM (5 ml) and washed with H₂O (2×3 ml). The organic phase was dried (Na₂SO₄), filtered and concentrated. The crude material was purified by flash chromatography on silica gel (Biotage SP 25M, Cy/EtOAc from 80/20 to 60/40) to afford the title compound D8 (0.11 g, 0.29 mmol, 52% yield).

UPLC: rt=0.92 min, peak observed: 374 (M+1). C₁₈H₂₆F₃N₃O₂ requires 373.

### Description 9

### N-{[(2S,5S)-5-methyl-2-piperidinyl]methyl}-5-(trifluoromethyl)-2-pyridinamine (D9)

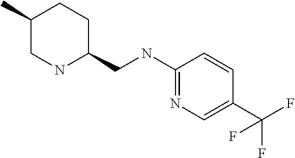

To a solution of 1,1-dimethylethyl (2S,5S)-5-methyl-2-({[5-(trifluoromethyl)-2-pyridinyl]amino}methyl)-1-piperidinecarboxylate (D8) (0.11 g, 0.29 mmol) in DCM (2 ml), TFA (1 ml) was added and the reaction mixture left under stirring for 2 h at room temperature. Volatiles were removed under reduced pressure and the residue eluted through a SCX column. Collected fractions gave the title compound D9 (0.068 g, 0.25 mmol, 86% yield). UPLC: rt=0.52 min, peak observed: 274 (M+1). C₁₃H₁₈F₃N₃ requires 273. ¹H-NMR [the SYN relative stereochemistry is derived from the stereochemistry of the previous intermediates] (400 MHz, CDCl₃) δ (ppm): 8.33 (s, 1H), 7.55 (dd, 1H), 6.44 (d, 1H), 5.35-5.55 (bs, 1H), 3.24-3.50 (m, 2H), 2.85-3.00 (m, 1H), 2.83 (dd, 1H), 2.65 (dd, 1H), 1.40-1.78 (m, 5H), 0.99 (d, 3H).

### Description 10

### 1,1-dimethylethyl (2S,55)-2-{[(5-chloro-2-pyridinyl)amino]methyl}-5-methyl-1-piperidinecarboxylate (D10)

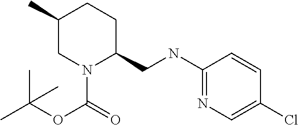

A solution of 1,1-dimethylethyl (2S,5S)-2-formyl-5-methyl-1-piperidinecarboxylate (D3) (1.40 g, 6.16 mmol) and 5-chloro-2-pyridinamine (0.95 g, 7.39 mmol) in DCE (25 ml) was stirred for 1 h at rt. Sodium triacetoxyborohydride (1.82 g, 8.62 mmol) was then added and the resulting reaction mixture left under stirring overnight at rt. A saturated Na₂CO₃ aqueous solution was added and the aqueous phase extracted with DCM (3×20 ml). The combined organic layers were dried (Na₂SO₄), filtered and concentrated. The crude oil was purified by flash chromatography on silica gel (Biotage SP 25M, from Cy/EtOAc 80/20 to 50/50) to afford the title compound D10 (0.86 g, 2.53 mmol, 41% yield).

UPLC: rt=0.82 min, peak observed: 340 (M+1). C₁₇H₂₆ClN₃O₂ requires 339.

### Description 11

### 5-chloro-N-{[(2S,5S)-5-methyl-2-piperidinyl]methyl}-2-pyridinamine (D11)

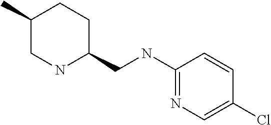

To a solution of 1,1-dimethylethyl (2S,5S)-2-{[(5-chloro-2-pyridinyl)amino]methyl}-5-methyl-1-piperidinecarboxylate (D10) (0.86 g, 2.53 mmol) in DCM (10 ml), TFA (3 ml, 38.90 mmol) was added and the resulting mixture stirred for 2 h. Volatiles were removed under reduced pressure and the residue eluted through a SCX column. Collected fractions gave the title compound D11 (0.60 g, 2.51 mmol, quantitative yield).

UPLC: rt=0.44 min, peak observed: 240 (M+1). C₁₂H₁₈ClN₃ requires 239.

¹H NMR [the SYN relative stereochemistry is derived from 1H,1H scalar coupling network. The ANTI stereoisomer is present at ca. 15%. Quantification of the ANTI isomer versus SYN is performed by integrating signals of methyl group of both stereoisomer] (400 MHz, CDCl₃) δ (ppm): 7.95-7.98 (m, 1H), 7.33 (dd, 1H), 6.42 (d, 1H), 5.93-6.05 (bs, 1H), 3.60-3.79 (m, 2H), 2.94-3.13 (m, 3H), 1.44-2.16 (m, 5H), 1.09 (d, 3H).

### EXAMPLES

### Example 1

### N-[((2S,5S)-1-{[2,6-bis(ethyloxy)phenyl]carbonyl}-5-methyl-2-piperidinyl)methyl]-5-bromo-2-pyridinamine (E1)

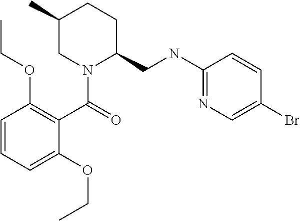

### Example 2

### 5-bromo-N-[((2S,5S)-5-methyl-1-{[2-(methyloxy)phenyl]carbonyl}-2-piperidinyl)methyl]-2-pyridinamine (E2)

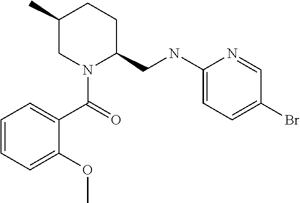

### Example 3

### 5-bromo-N-[((2S,5S)-1-{[2-(ethyloxy)phenyl]carbonyl}-5-methyl-2-piperidinyl)methyl]-2-pyridinamine (E3)

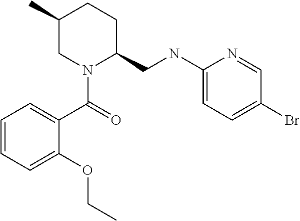

### Example 4

### 5-bromo-N—R(2S,5S)-1-{[3-fluoro-2-(methyloxy)phenyl]carbonyl}-5-methyl-2-piperidinyl)methyl]-2-pyridinamine (E4)

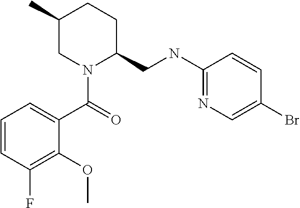

To a solution of 3-fluoro-2-(methyloxy)benzoic acid (4.7 g, 27.5 mmol) in dry DCM (78 ml), DIPEA (9.6 ml, 55 mmol) was added followed by TBTU (9.7 g, 30.3 mmol) at rt under nitrogen atmosphere. The mixture dissolved completely in about 10 minutes. After 30 minutes stirring, a solution of 5-bromo-N-{[(2S,55)-5-methyl-2-piperidinyl]methyl}-2-pyridinamine (D7) (7.8 g, 27.5 mmol) in dry DCM (78 ml) was added dropwise. The reaction mixture was left under stirring for 16 h and then diluted with DCM (200 ml) and a saturated NaHCO₃ aqueous solution (150 ml). The aqueous layer was back extracted with DCM (2×200 ml). The combined organic phases were washed with brine (1×100 ml), dried (Na₂SO₄), filtered and concentrated under reduced pressure to give a crude yellow oil. The material was purified by flash chromatography on silica gel (Biotage 75M, DCM/MeOH 98/2) and then the collected fraction purified again (Biotage 75M, Cy/EtOAc from 70/30 to 60/40) to afford the title compound E4 (8.9 g, 20.4 mmol, 74% yield) as a pale yellow gummy solid. MS: (ES/+) m/z: 436 (M+1, 100%) and 438 (M+1, 100%). UPLC: rt=3.41 min, peaks observed: 436 (M+1, 100%) and 438 (M+1, 100%). C₂₀H₂₃BrFN₃O₂ requires 435.

¹H NMR [the SYN relative stereochemistry is derived from the stereochemistry of the previous intermediates. Four conformers due to hindered rotation of the CO=group slowly exchange in solution (ratio ca. 50/20/15/15). Assignment is provided for one rotamer] (500 MHz, DMSO-d₆) δ (ppm): 8.01 (dd, 1H), 7.68 (d, 1H), 7.40 (dd, 1H), 7.07-7.14 (m, 1H), 6.89 (t, 1H), 6.62-6.68 (m, 1H), 6.36 (d, 1H), 4.79-4.99 (m, 1H), 4.38 (dd, 1H), 3.73 (s, 3H), 3.22-3.44 (m, 2H), 2.42-2.58 (m, 1H), 1.22-1.81 (m, 5H), 0.92 (d, 3H).

### Example 5

### 5-bromo-N-[((2S,5S)-1-{[4-chloro-2-(methyloxy)phenyl]carbonyl}-5-methyl-2-piperidinyl)methyl]-2-pyridinamine (E5)

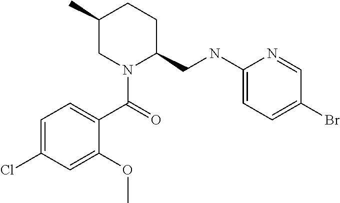

### Example 6

### 5-bromo-N-{[(2S,5S)-5-methyl-1-({2-[(trifluoromethyl)oxy]phenyl}carbonyl)-2-piperidinyl]methyl}-2-pyridinamine (E6)

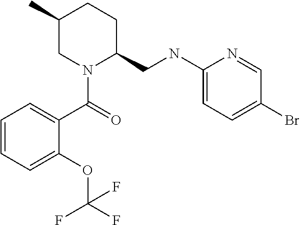

### Example 7

### 5-bromo-N-({(2S,5S)-5-methyl-1-[(2-methylphenyl)carbonyl]-2-piperidinyl}methyl)-2-pyridinamine (E7)

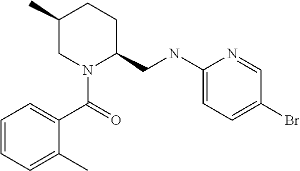

### Example 8

### N-[((2S,5S)-1-{[3-fluoro-2-(methyloxy)phenyl]carbonyl}-5-methyl-2-piperidinyl)methyl]-6-methyl-2-pyridinamine hydrochloride (E8)

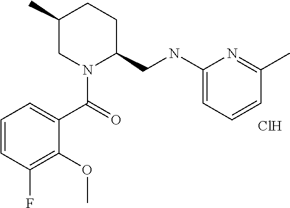

### Example 9

### 6-methyl-N-{[(2S,5S)-5-methyl-1-({2-[(trifluoromethyl)oxy]phenyl}carbonyl)-2-piperidinyl]methyl}-2-pyridinamine (E9)

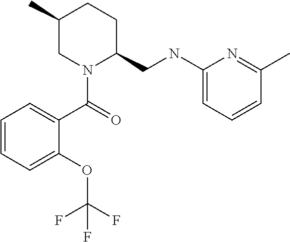

### Example 10

### 5-bromo-N-[((2S,5S)-1-{[5-fluoro-2-(methyloxy)phenyl]carbonyl}-5-methyl-2-piperidinyl)methyl]-2-pyridinamine (E10)

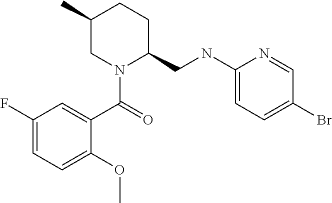

### Example 11

### 5-bromo-N-[((2S,5S)-1-{[4-fluoro-2-(methyloxy)phenyl]carbonyl}-5-methyl-2-piperidinyl)methyl]-2-pyridinamine (E11)

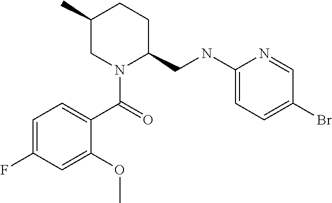

### Example 12

### 5-bromo-N-[((2S,5S)-1-{[2-fluoro-6-(methyloxy)phenyl]carbonyl}-5-methyl-2-piperidinyl)methyl]-2-pyridinamine (E12)

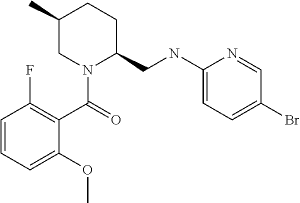

### Example 13

### N-{[(2S,5S)-1-({5-chloro-2-[(2-methylpropyl)oxy]phenyl}carbonyl)-5-methyl-2-piperidinyl]methyl}-6-methyl-2-pyridinamine hydrochloride (E13)

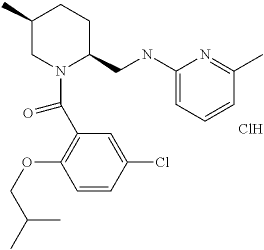

### Example 14

### N-[((2S,5S)-1-{[5-chloro-2-(ethyloxy)phenyl]carbonyl}-5-methyl-2-piperidinyl)methyl]-6-methyl-2-pyridinamine hydrochloride (E14)

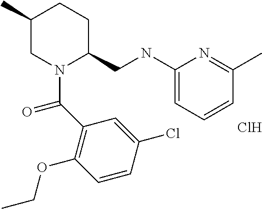

### Example 15

### 6-{[((2S,5S)-1-{[3-fluoro-2-(methyloxy)phenyl]carbonyl}-5-methyl-2-piperidinyl)methyl]amino}-3-pyridinecarbonitrile (E15)

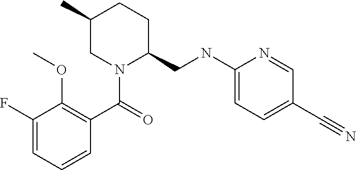

### Example 16

### N-[((2S,5S)-1-{[3-fluoro-2-(methyloxy)phenyl]carbonyl}-5-methyl-2-piperidinyl)methyl]-5-(trifluoromethyl)-2-pyridinamine (E16)

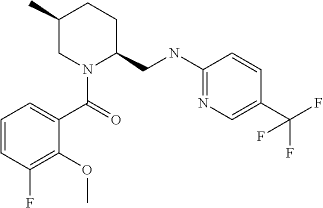

To a solution of 3-fluoro-2-(methyloxy)benzoic acid (14.9 mg, 0.088 mmol) in DMF (1 ml), DIPEA (0.077 ml, 0.44 mmol) and TBTU (32.9 mg, 0.10 mmol) were added and the reaction mixture left under stirring at rt for 1 h. A solution of N-{[(2S,5S)-5-methyl-2-piperidinyl]methyl}-5-(trifluoromethyl)-2-pyridinamine (D9) (20 mg, 0.073 mmol) in DMF (1 ml) was added at 0° C. The reaction mixture was left under stirring overnight at room temperature and then diluted with DCM and a saturated NaHCO₃ aqueous solution. The organic phase was washed with brine/ice, separated through a phase separator tube and the solvent removed under reduced pressure. The material was purified by flash chromatography on NH (Biotage SP4 12M, DCM/MeOH 98/2) and then the collected fraction purified again (Biotage 75M, from Cy 100 to Cy/EtOAc 40/60) to afford the title compound E16 (28.6 mg, 0.064 mmol, 88% yield). UPLC: rt=0.82 min, peak observed: 426 (M+1). C₂₁H₂₃F₄N₃O₂ requires 425.

¹H NMR [the SYN relative stereochemistry is derived from the stereochemistry of the previous intermediates. The ANTI stereoisomer is also present at ca. 5%. Quantification of the ANTI isomer versus SYN is performed by integrating signals of methyl group of both stereoisomer. For the SYN isomer, four conformers due to hindered rotation of the C═O group slowly exchange in solution (ratio ca. 44/26/18/12). Assignment is provided for one rotamer] (500 MHz, DMSO-d₆) δ (ppm): 8.27 (d, 1H), 7.83-8.00 (m, 1H), 6.79-7.67 (m, 3H), 6.36-6.66 (m, 2H), 4.91 (d, 1H), 4.42 (d, 1H), 3.58-3.94 (m, 5H), 3.00 (d, 1H), 1.08-1.84 (m, 5H), 0.94 (d, 3H).

### Example 17

### N-[((2S,5S)-1-{[3-fluoro-2-(methyloxy)phenyl]carbonyl}-5-methyl-2-piperidinyl)methyl]-6-(trifluoromethyl)-2-pyridinamine (E17)

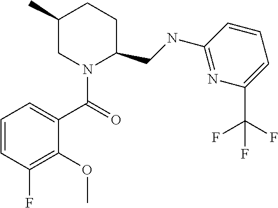

### Example 18

### N-[((2S,5S)-1-{[3-fluoro-2-(methyloxy)phenyl]carbonyl}-5-methyl-2-piperidinyl)methyl]-3-(trifluoromethyl)-2-pyridinamine (E18)

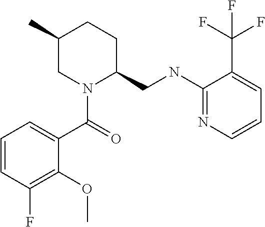

### Example 19

### 3,5-difluoro-N-[((2S,5S)-1-{[3-fluoro-2-(methyloxy)phenyl]carbonyl}-5-methyl-2-piperidinyl)methyl]-2-pyridinamine (E19)

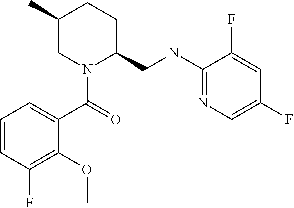

### Example 20

### N-[((2S,5S)-1-{[3-fluoro-2-(methyloxy)phenyl]carbonyl}-5-methyl-2-piperidinyl)methyl]-4-(trifluoromethyl)-2-pyridinamine (E20)

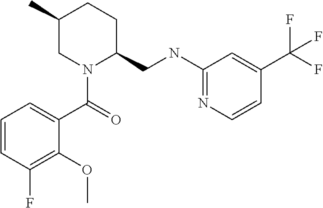

### Example 21

### N-[((2S,5S)-1-{[3-fluoro-2-(methyloxy)phenyl]carbonyl}-5-methyl-2-piperidinyl)methyl]-5-methyl-2-pyridinamine (E21)

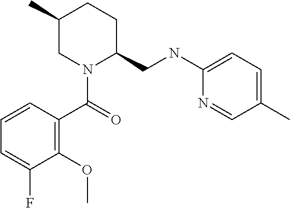

### Example 22

### 5-chloro-N-[((2S,5S)-1-{[3-fluoro-2-(methyloxy)phenyl]carbonyl}-5-methyl-2-piperidinyl)methyl]-2-pyridinamine (E22)

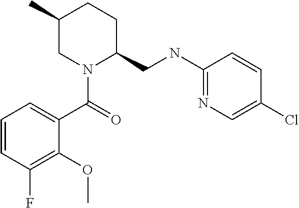

To a solution of 3-fluoro-2-(methyloxy)benzoic acid (0.47 g, 2.75 mmol) in DMF (2 ml), DIPEA (2.62 ml, 15.02 mmol) and TBTU (1.12 g, 3.50 mmol) were added and the reaction mixture left under stirring for 45 min. A solution of 5-chloro-N-{[(2S,55)-5-methyl-2-piperidinyl]methyl}-2-pyridinamine (D11) (0.60 g, 2.50 mmol) in DMF (1 ml) was added and the reaction mixture stirred for 2 h at rt. DMF was evaporated under vacuum and the residue was taken-up in DCM (10 ml) and washed with a saturated NaHCO₃ aqueous solution (10 ml). The organic phase was dried (Na₂SO₄), filtered and concentrated under reduced pressure. The material was purified by flash chromatography on silica gel (Biotage SP 350g SNAP column, from Cy to Cy/EtOAc 60/40). Collected fractions gave the title compound E22 (0.33 g, 0.84 mmol, 34% yield).

UPLC: rt=0.75 min, peak observed: 392 (M+1). C₂₀H₂₃ClFN₃O₂ requires 391.

¹H NMR [the SYN relative stereochemistry is derived from the stereochemistry of the previous intermediates. Conformers due to hindered rotation of the C═O group slowly exchange in solution. Assignment is provided for one rotamer] (400 MHz, DMSO-d₆) δ(ppm): 7.96 (dd, 1H), 7.62 (d, 1H), 7.35 (dd, 1H), 7.07-7.14 (m, 1H), 6.88 (t, 1 H), 6.62-6.68 (m, 1H), 6.41 (d, 1H), 4.83-4.97 (m, 1H), 4.43 (dd, 1H), 3.75 (s, 3 H), 3.22-3.44 (m, 2H), 2.42-2.58 (m, 1H), 1.22-1.81 (m, 5H), 0.92 (d, 3H).

### Example 23

### 5-fluoro-N-[((2S,5S)-1-{[3-fluoro-2-(methyloxy)phenyl]carbonyl}-5-methyl-2-piperidinyl)methyl]-4-methyl-2-pyridinamine (E23)

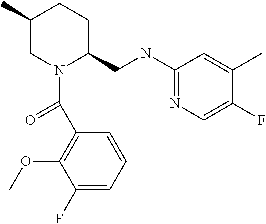

### Example 24

### 5-ethyl-N-[((2S,5S)-1-{[3-fluoro-2-(methyloxy)phenyl]carbonyl}-5-methyl-2-piperidinyl)methyl]-2-pyridinamine (E24)

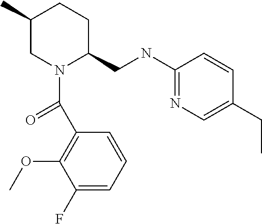

### Example 25

### 5-chloro-N-[((2S,5S)-1-{[2-(ethyloxy)-3-fluorophenyl]carbonyl}-5-methyl-2-piperidinyl)methyl]-2-pyridinamine (E25)

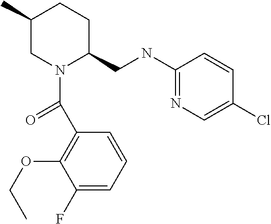

### Example 26

### N-[((2S,5S)-1-{[3-fluoro-2-(propyloxy)phenyl]carbonyl}-5-methyl-2-piperidinyl)methyl]-5-methyl-2-pyridinamine (E26)

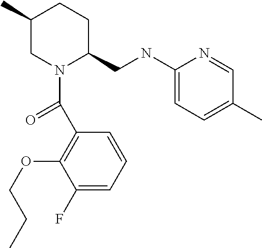

### Example 27

### Determination of Antagonist Affinity at Human Orexin-1 and 2 Receptors using FLIPR

### Cell Culture

Adherent Chinese Hamster Ovary (CHO) cells, stably expressing the recombinant human Orexin-1 (hOX1) or human Orexin-2 receptors (hOX2), were maintained in culture in Alpha Minimum Essential Medium (Gibco/Invitrogen, cat. no.; 22571-020), supplemented with 10% decomplemented foetal bovine serum (Life Technologies, cat. no. 10106-078) and 400 ug/mL Geneticin G418 (Calbiochem, cat. no. 345810). Cells were grown as monolayers under 95%:5% air:CO₂ at 37° C. and passaged every 3-4 days. The highest passage used was 25.

Measurement of [Ca²]ᵢ busing the FLIPR™

CHO-hOX1 or CHO-hOX2 cells were seeded into black clear-bottom 384-well plates at a density of 20,000 cells per well in culture medium as described above and maintained overnight (95%:5% air:CO₂ at 37° C.).

On the day of the experiment, culture medium were discarded and the cells washed three times with standard buffer (NaCl, 145 mM; KCl, 5 mM; HEPES, 20 mM; Glucose, 5.5 mM; MgCl₂, 1 mM; CaCl₂, 2 mM) added with Probenecid 2.5 mM.

The plates were then incubated at room temperature for 60 minutes in the dark with 1 μM FLUO-4AM dye to allow cell uptake of the FLUO-4AM, which is then converted by intracellular esterases to FLUO-4, which is unable to leave the cells.

After incubation, cells were washed three times with standard buffer to remove extracellular dye and 30 μL of buffer were left in each well after washing. Compounds of the invention were tested in a final assay concentration range from 1.66E-05M to 1.58E-11M.

Compounds of the invention were dissolved in dimethylsulfoxide (DMSO) at a stock concentration of 10 mM. These solutions are serially diluted with DMSO in a 384 compound plate and 1 μl of each dilution is transferred to the test compound plate. Just prior compounds addition to the cells, buffer (50 μL/well) was added to the 1 μL compound copy plate.

An agonist stimulus 384-well plate containing 50 μL/well of human orexin A (hOrexinA) was prepared just before using by diluting with buffer a stock plate: final concentration is equivalent to the calculated EC80 for hOrexinA. This value was obtained by testing hOrexinA in concentration response curve (at least 16 replicates) the same day of the experiment.

The loaded cells were then incubated for 10 min at 37° C. with test compound. The plates were then placed into a FLIPR™ (Molecular Devices, UK) to monitor cell fluorescence (λₑₓ=488 nm, λ_{EM}=540 nm) (Sullivan E, Tucker E M, Dale I L. Measurement of [Ca²⁺]ᵢ using the fluorometric imaging plate reader (FLIPR). In: Lambert DG (ed.), Calcium Signaling Protocols. New Jersey: Humana Press, 1999, 125-136). A baseline fluorescence reading was taken over a 5 to 10 second period, and then 10 μL of EC80 hOrexinA solution was added. The fluorescence was then read over a 45 minute period.

### Data Analysis

Functional responses using FLIPR were measured as peak fluorescence intensity minus basal fluorescence and expressed as a percentage of a non-inhibited Orexin-A-induced response on the same plate. Iterative curve-fitting and parameter estimations were carried out using a four parameter logistic model and Microsoft Excel (Bowen W P, Jerman J C. Nonlinear regression using spreadsheets. Trends Pharmacol. Sci. 1995; 16: 413-417). Antagonist affinity values (IC₅₀) were converted to functional pKᵢ values using a modified Cheng-Prusoff correction (Cheng Y C, Prusoff W H. Relationship between the inhibition constant (Kᵢ) and the concentration of inhibitor which causes 50 percent inhibition (IC₅₀) of an enzymatic reaction. Biochem. Pharmacol. 1973, 22: 3099-3108).

$$fpKi = - log \frac{( {IC}_{50} )}{{( 2 + {( \frac{[ agonist ]}{( {EC}_{50}} )}^{n} )}^{1 / n} - 1}$$

Where [agonist] is the agonist concentration, EC₅₀ is the concentration of agonist giving 50% activity derived from the agonist dose response curve and n=slope of the dose response curve. When n=1 the equation collapses to the more familiar Cheng-Prusoff equation.

Compounds of the Examples tested according to this method had fpKi values in the range from 7.2 to 10 at the human cloned orexin-1 receptor and from 5.9 to 8.6 at the human cloned orexin-2 receptor.

1. A compound of formula (I): 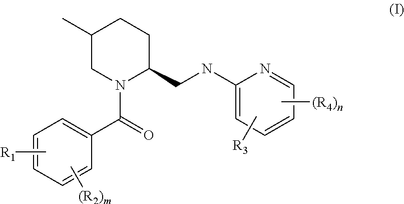
where:
R₁ is C₁₋₄alkyl, halo, C₁₋₄alkoxy, haloC₁₋₄ alkyl or haloC₁₋₄alkoxy;
R₂ is C₁₋₄alkyl, halo, C₁₋₄alkoxy, haloC₁₋₄alkyl or haloC₁₋₄alkoxy;
R₃ is C₁₋₄alkyl, halo, C₁₋₄alkoxy, haloC₁₋₄alkoxy or cyano;
R₄ is C₁₋₄alkyl, halo, C₁₋₄alkoxy, haloC₁₋₄alkyl or haloC₁₋₄alkoxy; and
m is 0 or 1;
n is 0 or 1;
provided that when m is 1 and one of R₁ and R₂ is trifluoromethyl, the other is not trifluoromethyl;
or a pharmaceutically acceptable salt thereof.

2. A compound according to claim 1 wherein the compound has the methyl at the 5 position on the piperidyl ring in the 5S isomeric form, or a pharmaceutically acceptable salt thereof.

3. A compound according to claim 1 where n is 0, or a pharmaceutically acceptable salt thereof.

4. A compound according to claim 1 where m is 1, or a pharmaceutically acceptable salt thereof.

5. A compound according to claim 1 where R₃ is halo, or a pharmaceutically acceptable salt thereof.

6. A compound according to claim 1 where R₃ is C₁₋₄alkyl, or a pharmaceutically acceptable salt thereof.

7. A compound according to claim 1 where R₁ is C₁₋₄alkyl, or a pharmaceutically acceptable salt thereof.

8. A compound according to claim 1 where R₁ is C₁₋₄alkoxy, or a pharmaceutically acceptable salt thereof.

9. A compound selected from the group consisting of:
N-[((2S,5S)-1-{[2,6-bis(ethyloxy)phenyl]carbonyl}-5-methyl-2-piperidinyl)methyl]-5-bromo-2-pyridinamine;
5-bromo-N-[((2S,5S)-5-methyl-1-{[2-(methyloxy)phenyl]carbonyl}-2-piperidinyl)methyl]-2-pyridinamine;
5-bromo-N-[((2S,5S)-1-{[2-(ethyloxy)phenyl]carbonyl}-5-methyl-2-piperidinyl)methyl]-2-pyridinamine;
5-bromo-N-[((2S,5S)-1-{[3-fluoro-2-(methyloxy)phenyl]carbonyl}-5-methyl-2-piperidinyl)methyl]-2-pyridinamine;
5-bromo-N-[((2S,5S)-1-{[4-chloro-2-(methyloxy)phenyl]carbonyl}-5-methyl-2-piperidinyl)methyl]-2-pyridinamine;
5-bromo-N-{[(2S,5S)-5-methyl-1-({2-[(trifluoromethyl)oxy]phenyl}carbonyl)-2-piperidinyl]methyl}-2-pyridinamine;
5-bromo-N-({(2 S,5S)-5-methyl-1-[(2-methylphenyl)carbonyl]-2-piperidinyl}methyl)-2-pyridinamine;
N-[((2S,5S)-1-{[3-fluoro-2-(methyloxy)phenyl]carbonyl}-5-methyl-2-piperidinyl)methyl]-6-methyl-2-pyridinamine hydrochloride;
6-methyl-N-{[(2S,5S)-5-methyl-1-({2-[(trifluoromethyl)oxy]phenyl}carbonyl)-2-piperidinyl]methyl}-2-pyridinamine;
5-bromo-N-[((2S,5S)-1-{[5-fluoro-2-(methyloxy)phenyl]carbonyl}-5-methyl-2-piperidinyl)methyl]-2-pyridinamine;
5-bromo-N-[((2S,5S)-1-{[4-fluoro-2-(methyloxy)phenyl]carbonyl}-5-methyl-2-piperidinyl)methyl]-2-pyridinamine;
5-bromo-N-[((2S,5S)-1-{[2-fluoro-6-(methyloxy)phenyl]carbonyl}-5-methyl-2-piperidinyl)methyl]-2-pyridinamine;
N-{[(2S,5S)-1-({5-chloro-2-[(2-methylpropyl)oxy]phenyl}carbonyl)-5-methyl-2-piperidinyl]methyl}-6-methyl-2-pyridinamine hydrochloride;
N-[((2S,5S)-1-{[5-chloro-2-(ethyloxy)phenyl]carbonyl}-5-methyl-2-piperidinyl)methyl]-6-methyl-2-pyridinamine hydrochloride;
6-{[((2S,5S)-1-{[3-fluoro-2-(methyloxy)phenyl]carbonyl}-5-methyl-2-piperidinyl)methyl]amino}-3-pyridinecarbonitrile;
N-[((2S,5S)-1-{[3-fluoro-2-(methyloxy)phenyl]carbonyl}-5-methyl-2-piperidinyl)methyl]-5-(trifluoromethyl)-2-pyridinamine;
N-[((2S,5S)-1-{[3-fluoro-2-(methyloxy)phenyl]carbonyl}-5-methyl-2-piperidinyl)methyl]-6-(trifluoromethyl)-2-pyridinamine;
N-[((2S,5S)-1-{[3-fluoro-2-(methyloxy)phenyl]carbonyl}-5-methyl-2-piperidinyl)methyl]-3-(trifluoromethyl)-2-pyridinamine;
3,5-difluoro-N-[((2S,5S)-1-{[3-fluoro-2-(methyloxy)phenyl]carbonyl}-5-methyl-2-piperidinyl)methyl]-2-pyridinamine;
N-[((2S,5S)-1-{[3-fluoro-2-(methyloxy)phenyl]carbonyl}-5-methyl-2-piperidinyl)methyl]-4-(trifluoromethyl)-2-pyridinamine;
N-[((2S,5S)-1-{[3-fluoro-2-(methyloxy)phenyl]carbonyl}-5-methyl-2-piperidinyl)methyl]-5-methyl-2-pyridinamine;
5-chloro-N-[((2S,5S)-1-{[3-fluoro-2-(methyloxy)phenyl]carbonyl}-5-methyl-2-piperidinyl)methyl]-2-pyridinamine;
5-fluoro-N-[((2S,5S)-1-{[3-fluoro-2-(methyloxy)phenyl]carbonyl}-5-methyl-2-piperidinyl)methyl]-4-methyl-2-pyridinamine;
5-ethyl-N-[((2 S,5S)-1-{[3-fluoro-2-(methyloxy)phenyl]carbonyl}-5-methyl-2-piperidinyl)methyl]-2-pyridinamine;
5-chloro-N-[((2S,5S)-1-{[2-(ethyloxy)-3-fluorophenyl]carbonyl}-5-methyl-2-piperidinyl)methyl]-2-pyridinamine; and
N-[((2S,5S)-1-{[3-fluoro-2-(propyloxy)phenyl]carbonyl}-5-methyl-2-piperidinyl)methyl]-5-methyl-2-pyridinamine,
or a pharmaceutically acceptable salt thereof.

10-18. (canceled)

19. A method for the treatment of a disease or disorder where an antagonist of a human orexin receptor is required, in a subject in need thereof, comprising administering to said subject an effective amount of a compound as defined in claim 1, or a pharmaceutically acceptable salt thereof, where the disease or disorder is a sleep disorder, a depression or mood disorder, an anxiety disorder, a substance-related disorder or a feeding disorder.

20. (canceled)

21. A method according to claim 20 where the disease or disorder is a sleep disorder.

22. A method according to claim 21 where the sleep disorder is selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

23. A pharmaceutical composition comprising a) the compound as defined in claim 1, or a pharmaceutically acceptable salt thereof, and b) one or more pharmaceutically acceptable carriers.
